(19) United States
(12) Patent Application Publication
(10) Pub. No.: US 2004/0013676 A1
(43) Pub. Date: Jan. 22, 2004

(54) Pharmaceutical compositions comprising dendritic cells for immunotherapy of autoimmune disease and treatment methods using the same

(76) Inventors: Yong-Soo Bae, Deajon Metropolitan City (KR); Choon-Ju Jeon, Daejon Metropolitan City (KR); Yoon Lee, Daejon Metropolitan City (KR); Ki-Duk Song, Daejon Metropolitan City (KR); Chang-Hyun Kim, Daejon Metropolitan City (KR); Il-Soo Kim, Seoul (KR); Hyun-Pil Cho, Kwangmyung-shi (KR); Seon-Gil Do, Kwangmyung-shi (KR); Hye-Jung Nam, Seoul (KR)

Correspondence Address: NIXON & VANDERHYE, PC, 1100 N GLEBE ROAD, 8TH FLOOR, ARLINGTON, VA, 22201-4714, US

(21) Appl. No.: 10/312,512

(22) PCT Filed: Apr. 26, 2002
(86) PCT No.: PCT/KR02/00780

(30) Foreign Application Priority Data
Apr. 27, 2001 (KR) 2001/23098

(51) Int. Cl. A61K 39/00; C12N 5/08

(52) U.S. Cl. 424/184.1; 435/372; 424/93.7

(57) ABSTRACT

The present invention relates to a pharmaceutical composition for immunotherapy of autoimmune disease, which comprises (a) a therapeutically effective dose of maturated dendritic cells and (b) a pharmaceutically acceptable carrier and a method for immunotherapy of autoimmune disease.

### BACKGROUND OF THE INVENTION

1. Field of the Invention

The present invention relates to pharmaceutical compositions for immunotherapy of autoimmune diseases and particularly to pharmaceutical compositions comprising dendritic cells (DC) for immunotherapy of autoimmune diseases and their uses.

2. Description of the Related Art

Autoimmunity results from a breakdown in the regulation in the immune system resulting in an inflammatory response directed at self-antigens and tissues. The autoimmune diseases involving the destruction of self-antigen by T lymphocytes include the multiple sclerosis, insulin-dependent diabetes mellitus (also referred to as “IDDM” or “type I DM”) and the rheumatoid arthritis, etc (K J Johnson et al., Immunopathology in Pathology, pp.104-153(1999)).

Insulin-dependent diabetes mellitus resulting from the destruction of β-cells in the pancreatic islet by autoimmune T lymphocytes, may be diagnosed through the presence of the antibody against β-cell specific antigen such as glutamate decarboxylase (GAD65) (Lohmann et al., Lancet, 343:1607(1994); Yoon et al., Science, 284:1183-1187(1999)) or the antibody against insulin (Williams et al., J. Autoimmun. 13(3):357-363(1999); and Yu et al., PNAS. USA, 97(4):1701-1706(2000)); however, the comprehension on the precise cause and immunologic mechanism as well as the genetic factors of type I DM are remained to be elucidated and the reliable therapy has not been developed.

Although the NOD (non-obese diabetic) mice (Makino et al., Exp. Anim., 29:1-13(1980)) and BB (BioBreeding) rat (Like et al., Science, 216:644-646(1982)) have been developed as animal models for human type I DM, the etiological mechanism or therapeutic performance has not been fully understood.

Green et al. (Immunity, 9:733-743(1998)), suggested the involvement of potential antigen presenting cell, DC, in type I DM; more particularly in the islet specific TNF-α transgenic NOD mice in which local expression of TNF-α in islet initiates the insulitis, it is suggested that DC migrate into inflammatory site and thereafter induce the strong activation of autoimmune T lymphocytes to islet-specific antigen to accelerate insulitis, thereby leading to DM in the end.

However, Green et al. could not elucidate the mechanism of DM development in normal NOD mice. Meanwhile, it is reported that DC presenting the antigen acquired effectively from apoptotic cells activate cytotoxic T lymphocytes (CTLs) by cross-priming (Albert et al., Nature, 392:86-89(1998)). In addition, Rovere et al. elucidated the relevance of DC in DM development by the comparison of normal mouse with NOD mouse (J. Immunol., 161:4467-4471(1998); and J. Leuk. Biol., 66:345-349(1999)). According to Rovere et al., in normal mouse, the regeneration of islet β-cells is accompanied with clearing of the apoptotic cells mainly by macrophages and in part by immature DC, and these DC induce clonal deletion or anergy of islet antigen-specific CTLs resulting in the tolerance to islet antigen. On the contrary, in NOD mouse, abundant immature DC participate in the deletion of apoptotic cells owing to the excess of the apoptotic cells, and some DC matured during the deletion of apoptotic cells activate the islet antigen-specific CTLs leading to the-destruction of islet and to DM development.

The fact that B cells play their roles as antigen presenting cell (APC) in early insulitis was investigated using B cell deficient NOD (B-NOD) mice (Akashi et al., Int. Immunol., 9:1159-1164(1997); Noorchashm et. al., Diabetes, 46:941-946(1997); and Serreze et al., J. Exp. Med., 184:2409-2053(1996)). However, there are several reports indicating the major relevance of DC in early DM rather than B cells; Green et al. revealed the occurrence of DM under absence of B cells in the TNF-α-NOD mouse model (Curr. Opinion. Immunol., 11:663-669(1999)), Delon et al. indicated that activated DC are 10 times more potent in DM development than the same amount of activated B cells (J. Exp. Med., 188:1473-1484(1988)) and Voorbij et al. showed the infiltration of high number of DC into islet of NOD mouse or BB rat in the course of the early DM (Diabetes, 38:1623-1629(1989); Jansen et al., Diabetes, 43:667-675(1994); Dahlen et al., J. Immunol., 160:3585-3593 (1998); Papaccio et al., J. Cell Biochem., 74:447-457(1999); Rosmalen et al., Lab Invest., 80:23-30 (2000); and Rosmalen et al., Lab Invest., 80:769-777 (2000)). In addition, Ludwig et al. reported that EAE (Experimental Autoimmune Encephalitis), an animal model for autoimmune brain diseases, could be initiated by expressing self-antigen or DC pulsed with self-antigen transfer (Ludwig et al., J. Exp. Med., 188:1493-1501(1998); and Dittel et al., J. Immunol., 163:32-39(1999)). These investigations may be supported pivotal role of DC in DM development.

Among recent breakthroughs relating to researches on DC therapy of DM, the most conspicuous one is the reduction of DM occurrence in NOD mice (from 70% to 26.3%) by intraperitoneal injection into 1-4 wk old NOD mice with the IFN-γ treated low-adhesive spleen DC which are separated from spleen of normal NOD mice (Shinomiya et al., Clin. Exp. Immunol., 117:38-43(1999)). Shinomiya et al. also confirmed the similar preventive effect of ICR splenic DC when they are transferred to NOD mice. In another experiment, Shinomiya et al. observed that there was no DM occurrence in all 6 NOD mice for 30 wk if the DC were injected twice at the time point of 4 wk and 6 wk after birth. In spite of the strong preventive effect of DC in 1-4 wk old NOD mice, the DC injection did not work in 6 wk old or older NOD mice. Clare-Salzler et al. (Clare-Salzler et al., J. Clin, Invest., 90:741-748(1992)) showed that NOD mice injected with the pancreatic lymph node DC exhibited significant DM-prophylactic effect. These results suggest that DC could be used as prevention purpose for DM.

Furthermore, the human γ-globulin (HGG) treated spleen DC, which were isolated from NOD mice, showed the DM-prophylactic effect in which 11 NOD mice among 12 NOD mice showed DM-prophylaxis for 25 wk, and the islet culture of DC-injected NOD mice was proved to contain decreased level of IFN-γ and TNF-α as well as increased level of IL-4 and IL-10 (Papaccio et al., Endocrinology, 141:1500-1505(2000)). On the contrary, HGG-untreated DC did not show any DM-prophylactic effect. These results suggest that the control of abnormal immune response is possible by appropriate activation of DC and also suggest the abnormal activation of DC as an etiological cause of DM in type I DM patients or NOD mice. Actually, the possible cause of antigen-presenting cells (APC) in type I DM patients (Jansen et al. Lancet, 345:491-492 (1995); and Takahashi et al., J, Immunol., 161:2629-2635 (1999)) and NOD mice (Serreze et al., J. Immunol. , 150:2534-2543 (1993)) was suggested by several researchers. The failure of regulation on T lymphocytes by the immature DC was suggested as a cause of DM (Delemarre et al., J. Immunol. , 162:1795-1801 (1999)). In addition, Lee et al. indicated that bone marrow-derived DC of NOD mouse poorly matured to myeloid DC and showed lower expression of MHC type II, co-stimulatory molecules (B7-1 and B7-2), CD40, and lower level of IL-12 secretion compared to C57BL/6 mice (J. Korean Med. Sci., 15:217-223 (1999)). These results are supported by the suggestion of Takahashi et al. (J. Immunol., 161:2629-2635(1999)) showing the maturated monocyte-derived DC (Mo-DC) do not activate T lymphocytes efficiently and one of the reasons thereto may be suggested to be the low expression of B7 molecules in type I DM patients.

Although the above-described results indicate that the pivotal relevance of DC in type I DM and the prophylactic possibility to type I DM with DC, the therapeutic application of DC on DM has remained to be investigated.

Currently, the drugs for treating or alleviating rheumatoid arthritis include methotrexate, azathioprine, cyclophosphamide and corticosteroid (Johnson CJ et al., Ann. Pharmacother., 35 (4):464-471(2001); and Seymour HE et al., Br. J. Clin. Pharmacol., 51(3):201-208(2001)). However, the described drugs are incapable of preventing the destruction of the joint efficiently and have several side effects as well.

U.S. Pat. No. 6,007,821 discloses methods and compositions for the treatment of autoimmune disease, which include gp96, hsp90 and hsp70. U.S. Pat. No. 6,098,631 discloses methods for treating autoimmune disease using inhibitors of the sphingomyelin signal transduction pathway. In addition, U.S. Pat. No. 6,184,253 discloses methods for treating autoimmune disease comprising administering to a patient in need thereof a therapeutically effective amount of toremifene or a pharmaceutically acceptable salt thereof.

Throughout this application, various patents and publications are referenced and citations are provided in parentheses. The disclosure of these patents and publications in their entitles are hereby incorporated by references into this application in order to more fully describe this invention and the state of the art to which this invention pertains.

### SUMMARY OF THE INVENTION

The present inventors have isolated certain dendritic cell subsets from mouse spleen and have discovered that the dendritic cell subsets treated with an appropriate cytokine for maturation have shown the alleviating or removing effect of autoimmune response, thus accomplishing this invention.

Accordingly, it is an object of this invention to provide pharmaceutical compositions for immunotherapy of autoimmune diseases.

It is another object of this invention to provide methods for immunotherapy of autoimmune diseases.

Other objects and advantages of the present invention will become apparent from the detailed description to follow taken in conjugation with the appended claims and drawings.

### BRIEF DESCRIPTION OF THE DRAWINGS

FIGS. 1a-1f represent summarization of isolation method of specific dendritic cell subsets used in this invention.

FIGS. 2a-2d represent FACS results demonstrating expression pattern of surface proteins of dendritic cells isolated in Examples.

FIG. 3 represents yields of CD11b⁻/CD8a⁺/CD86 dendritic cell according to isolation method.

FIG. 4 shows viability of isolated CD11b⁻/CD8a⁺/CD86⁻ dendritic cells (DC) cultured in presence of IFN-γ.

FIG. 5a shows the changes of blood glucose level in NOD mice with ageing.

FIG. 5b represents diabetes development pattern in NOD mice with ageing.

FIG. 6a represents evaluation on initial response under single injection of DC. The numeric values denote a percentage of NOD mice showing initial response; and the numeral in parenthesis denotes the total number of NOD mice tested.

FIG. 6b represents the initial response and the duration of normoglycemia in NOD mice injected with DC varying based on the type of DC subsets.

FIG. 6c demonstrates the duration of normoglycemia in NOD mice injected and boosted with DC varying based on the type of DC subsets.

FIG. 7 shows hematoxylin and eosin staining results of islet demonstrating the therapeutic efficacy of DC on diabetes mellitus.

FIG. 8 shows immunohistochemical staining results of insulin demonstrating the therapeutic efficacy of DC on diabetes mellitus.

FIG. 9 represents the results of in vivo migration of DC and autoimmune T lymphocytes.

FIG. 10a shows the changes in IL-4 and IFN-γ contents in pancreatic lymph node cells, which are isolated from NOD mouse with early diabetes mellitus, treated with islet antigen.

FIG. 10b shows the changes in IL-4 and IFN-γ contents in pancreatic lymph node cells, which are isolated from diabetes mellitus-cured NOD mouse demonstrating the conversion of immune reaction.

FIG. 11 shows morphological changes of CD11b⁻/CD8a⁺ DC after treatment of IFN-γ.

FIG. 12 shows morphological changes of CD11c⁻ and CD11c⁺ DC isolated from human peripheral blood.

### DETAILED DESCRIPTION OF THIS INVENTION

In one aspect of this invention, there is provided a pharmaceutical composition for immunotherapy of autoimmune disease comprising (a) a therapeutically effective dose of maturated denderitic cells; and (b) a pharmaceutically acceptable carrier.

The term used herein “maturated dendritic cell” means the maturated dendritic cells developed in vitro or ex vivo by treating appropriate cytokine on the immature dendritic cells having no surface co-stimulatory molecules (e.g., for mouse, B7 molecules, CD80 or CD86). The term, dendritic cells or dendritic cell is referred to “DC” hereinafter.

By the term “treatment” or “treating”, it is meant to (a) a prophylaxis or prevention of autoimmune disease from occurring in an animal, preferably mammal, more preferably, human which may be predisposed to the disease but has not yet been diagnosed as having it; (b) an inhibition of autoimmune disease, i.e., arresting its development; and (c) an alleviation or relief of autoimmune disease.

In this invention, the autoimmune diseases therapeutically applicable by the DC include any disease or disorder caused by autoimmune response comprising type I DM, rheumatoid arthritis, multiple sclerosis, systemic lupus erythematosus, Sjogren's syndrome, scleroderma, polymyositis, chronic active hepatitis, mixed connective tissue disease, primary biliary cirrhosis, pernicious anemia, autoimmune thyroiditis, idiopathic Addison's disease, vitiligo, gluten-sensitive enteropathy, Graves' disease, myasthenia gravis, autoimmune neutropenia, diopathic thrombocytopenia purpura, cirrhosis, pemphigus vulgaris, autoimmune infertility, Goodpasture's disease, bullous pemphigoid, discoid lupus, ulcerative colitis and dense deposit disease. Preferably, the applicable diseases or disorders of the pharmaceutical composition of this invention are type I DM or rheumatoid arthritis.

As described above, although the DM-prophylactic effect of DC has been made public, the therapeutic application of maturated DC on type I DM has remained to be tried. Therefore, the discovery of this invention, i.e., the successful therapeutic application of maturated DC on type I DM is novel and surprising. In addition, the possible therapy of type I DM with DC also suggests the immunotherapy of other autoimmune diseases with DC.

For example, in the rheumatoid arthritis represented by a systemic chronic autoimmune disease, inflammation in joint continually infiltrates into cartilage and osteoid tissue resulting in bone corrosion. Type II collagen, a major constituent of joint, is well-known antigen causing arthritis and there is a publication showing that type II collagen causes rheumatoid arthritis in mice having specific MHC antigen (LK Myers et al., Life Sci., 19:1861-1878(1997)). In rheumatoid patient, the amount of cytokines secreted from macrophage or fibroblast is increased, and Th1 specific cytokines including IFN-γ and IL-2 are also accentuated. The Th1 specific cytokines are known to exacerbate arthritis contrary to the arthritis-prophylactic effect of Th2 cytokines comprising IL-4 and IL-10. Furthermore, SH Kim et al. showed that injecting into leg of artificially arthritis-induced mouse viral vectors expressing Th2 cytokines, IL-4 or IL-10, provided treatment effect for arthritis even in non-injected leg as well as injected one (S H Kim, et al., J. Immunol., 166: 3499-3505 (2001)). These findings suggest that both rheumatoid arthritis and type I DM have the same causing and therapeutic mechanism of autoimmune response while having difference in view of distinct MHC antigen being responsible for rheumatoid arthritis.

On the basis of these grounds, this invention also employs DC useful for DM therapy in order to treat rheumatoid arthritis.

In preferred embodiment of this invention, maturated DC can be prepared by isolating mature DC directly from animal body or by maturating the isolated immature DC using treatment with suitable cytokines. In addition, DC employed in this invention can be isolated from animal, preferably mammal and more preferably human organ, tissue, bone marrow or blood.

The suitable cytokines in maturation of DC comprise IFN-γ, TNF-α, TGF-β, IL-4 and IL-10, and IFN-γ is the most preferable. The IFN-γ for maturating DC is employed in the amount of 10²-10⁶ DC/unit, more preferably, 10⁴-10⁵ DC/unit.

According to the present invention, the therapeutic efficacy of maturated DC on autoimmune disease is manifested through the inhibition of activity of autoimmune T lymphocytes, which is accomplished by conversion of autoimmune Th1 lymphocyte into Th2 lymphocyte or by generation of new Th2 lymphocyte.

While allogeneic or syngeneic DC are applicable in this invention, syngeneic DC are preferred due to their notable therapeutic efficacy on autoimmune diseases. The term used herein, “allogeneic DC” refers to the DC isolated from donor whose major histocompatibility is different from recipient. For example, in case of using NOD mice (H-2b^{IA-g7}) as recipient, the DC isolated from BalB/c mice (H-2d; C3H, H-2k) is considered to be allogeneic DC.

According the preferred embodiment of the present invention, both lymphoid and myeloid DC are suitable and lymphoid DC is more preferable in view of therapeutic efficacy. The term used herein “lymphoid DC” refers to DC with the same hematopoietic lineage as T cells and B cells, e.g., for mouse, DC with CD11b⁻/CD8a⁺ phenotype of surface protein and the term “myeloid DC” refers to DC with the same hematopoietic lineage as monocyte and macrophage, e.g., for mouse, CD11b⁺/CD8a⁻ phenotype of surface protein.

According to the most preferred embodiment of this invention, the pharmaceutical composition comprises human DC subset showing surface phenotype of CD11c⁻/CD4a⁺. CD11c⁻/CD4a⁺ DC are maturated into CD11c⁻/CD4a⁺/CD86⁺ DC by treating with suitable cytokine such as IFN-γ. The mouse DC subset corresponding to CD11c⁻/CD4a⁺ human DC subset is CD11b⁻/CD8a⁺ DC, which is demonstrated in Examples.

According to the most preferred embodiment, this invention provides a pharmaceutical composition for immunotherapy of autoimmune diseases, which comprises (a) a therapeutically effective dose of maturated DC prepared by pretreatment with IFN-γ and (b) a pharmaceutically acceptable carrier.

In the pharmaceutical compositions of this invention, the pharmaceutically acceptable carrier may be conventional one for formulation, including lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methylhydroxy benzoate, propylhydroxy benzoate, talc, stearic acid, magnesium and mineral oil, but not limited to. The pharmaceutical compositions of this invention, further may contain wetting agent, sweetening agent, emulsifying agent, suspending agent, preservatives, flavors, perfumes, lubricating agent, or mixtures of these substances. In addition, the pharmaceutical compositions of this invention can be readily prepared since the pharmaceutical compositions comprise a physiological saline suspension serving as carrier.

The pharmaceutical compositions of this invention may be administered orally or parenterally, and the parenteral administration comprises intravenous injection, subcutaneous injection, intramuscular injection and intraperitoneal injection. Furthermore, the administration mode may be varied depending on diseases, for example, the intraperitoneal injection can be preferably employed for type I DM since the injected DC can migrate into pancreas without further dilution. In addition to this, the intravenous injection is recommended for rheumatoid arthritis and the most preferable administration mode is local injection into joint region directly.

The correct dosage of the pharmaceutical compositions of the invention will vary according to the particular formulation, the mode of application, age, body weight and sex of the patient, diet, time of administration, condition of the patient, drug combinations, reaction sensitivities and severity of the disease. It is understood that the ordinary skilled physician will readily be able to determine and prescribe a correct dosage of this pharmaceutical compositions. An exemplary dosage for type I DM is 10⁶-10⁷ maturated DC in the intraperitoneal injection, and for rheumatoid arthritis 10⁵-10⁶ maturated DC in the articular injection.

According to the conventional techniques known to those skilled in the art, the pharmaceutical compositions of this invention can be formulated with pharmaceutical acceptable carrier and/or vehicle as described above, finally providing several forms including a unit dosage form. Non-limiting examples of the formulations include, but not limited to, a solution, a suspension or an emulsion, an extract, an elixir, a powder, a granule, a tablet, a capsule, emplastra, a liniment, a lotion and an ointment.

In another aspect of the present invention, there is provided a method for immunotherapy of autoimmune diseases comprising the steps of (a) preparing maturated DC; and (b) administering into mammals a pharmaceutical composition containing (i) a therapeutically effective dose of the maturated DC and (ii) a pharmaceutically acceptable carrier.

The present method may be characterized by employing maturated DC described above. Therefore, the common descriptions between method and pharmaceutical composition of this invention are abbreviated in order to avoid the complexity of this specification leading to undue multiplicity.

The autoimmune diseases treated by this method include any disease or disorder caused by autoimmune response comprising type I DM, rheumatoid arthritis, multiple sclerosis, systemic lupus erythematosus, Sjogren's syndrome, scleroderma, polymyositis, chronic active hepatitis, mixed connective tissue disease, primary biliary cirrhosis, pernicious anemia, autoimmune thyroiditis, idiopathic Addison's disease, vitiligo, gluten-sensitive enteropathy, Graves' disease, myasthenia gravis, autoimmune neutropenia, diopathic thrombocytopenia purpura, cirrhosis, pemphigus vulgaris, autoimmune infertility, Goodpasture's disease, bullous pemphigoid, discoid lupus, ulcerative colitis and dense deposit disease. Preferably, the applicable diseases or disorders of this method are type I DM or rheumatoid arthritis.

In preferred embodiment of this invention, maturated DC can be prepared by isolating mature DC directly from animal body or by maturating the isolated immature DC using treatment with suitable cytokines. In addition, DC employed in this invention can be isolated from animal, preferably mammal and more preferably human organ, tissue, bone marrow or blood.

In the therapeutic method of this invention, both syngeneic and allogeneic DC can be used and allogeneic DC are preferred. Lymphoid DC are plausible for this therapeutic method, and CD11c⁻-/CD4⁺ DC subset is more plausible. CD11c⁻/CD4a⁺ DC are maturated into CD11c⁻/CD4a⁺/CD86⁺ DC by treating with IFN-γ.

In the therapeutic method of this invention, the single administration of DC is effective, however, the additional boosting is preferable after the first injection. Moreover, the preferable candidate for boosting is trans-allo-DC. The term “trans-allo-DC” means DC isolated from a donor whose MHC antigen is different from that used in the first administration.

According to the present invention, the step of administering is performed orally or parenterally, and the parenteral administration comprises intravenous injection, subcutaneous injection, intramuscular injection and intraperitoneal injection. Furthermore, the administration mode may be varied depending on diseases, for example, the intraperitoneal injection can be preferably employed for type I DM. In addition, the intravenous injection is recommended for rheumatoid arthritis and the most preferable administration mode is local injection into joint region directly.

According to Examples of this invention, the development of type I DM is classified into 6 steps according to symptoms as below (Eisenbarth, New Engl. J. Med. 314:1360-1368(1986)): (a) Stage I characterized by showing an essential genetic susceptibility without sufficient condition for development of DM; (b) Stage II represented by triggering the activation of autoimmune response against islet β-cells; (c) Stage III characterized by showing the reduction of islet β-cells, the abnormal immunity such as the occurrence of autoimmune antibody against insulin and the cytoplasmic antigen in islet; (d) Stage IV characterized by showing the progressive reduction of islet β-cells leading to reduction of insulin secretion in spite of showing normal blood glucose level; (e) Stage V represented by showing apparent symptom of DM (hyperglycemia) and destruction of around 90% islet β-cells requiring insulin-treatment for patient's survival; and (f) Stage VI represented by destruction of all islet β-cells and absence of C-peptide in blood. In accordance with the developing step of DM, the compositions and methods of this invention may be applicable to all the stages of the development, which gives rise to therapeutic efficacy. Preferably, this invention is applied to patients in Stage II-V and is significantly efficient even in Stage V.

The following specific examples are intended to be illustrative of the invention and should not be construed as limiting the scope of the invention as defined by appended claims.

### EXAMPLE I

### Isolation of CD11b⁻/CD8a⁺/CD86⁻ Dendritic Cells (DC) from Mouse Spleen

I-1: Isolation of CD11b⁻/CD8a⁺/CD86⁻ Dendritic Cells (DC) (Method 1)

The spleens were removed from ICR or BalB/c mice(Daehan Biolink, Korea), rinsed with PBS in Petridish and then collagenase solution (100 units/ml in PBS: Sigma Type IV, USA) was injected into the rinsed spleens with a syringe. After 5 min reaction, the spleens were chopped with a syringe needles for exudation of spleen cells into collagenase solution. Remained spleen cells were separated into collagenase solution by reacting the spleens with collagenase solution in a 50 ml tube for 25 min at RT (room temperature).

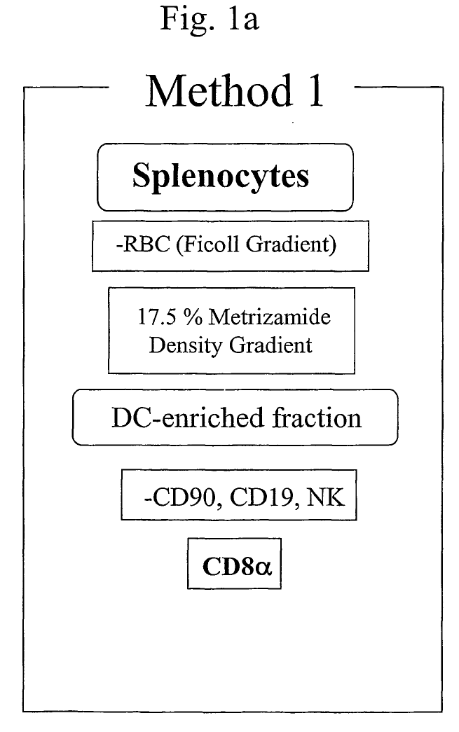

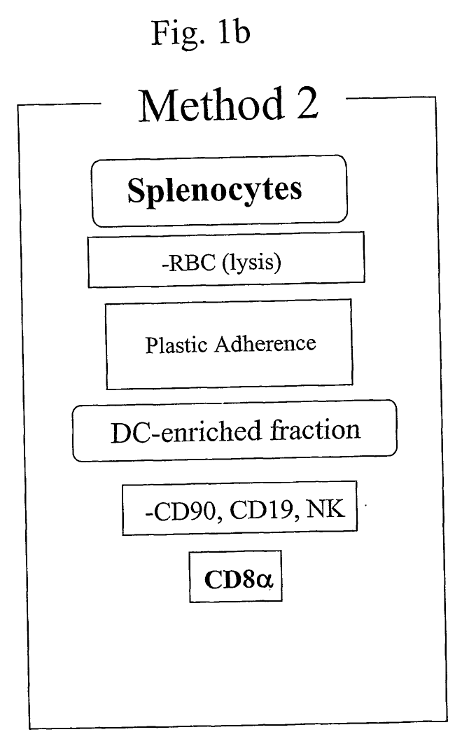

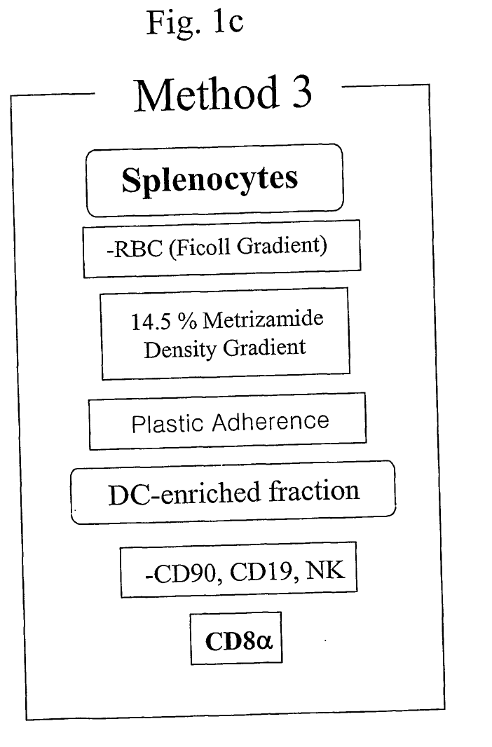

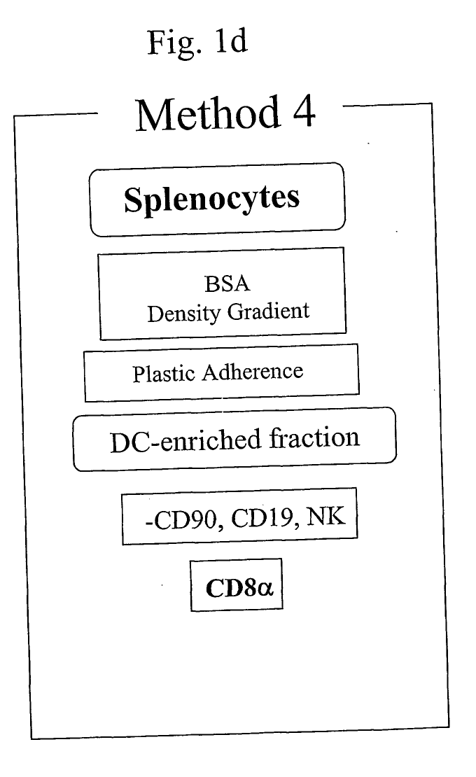

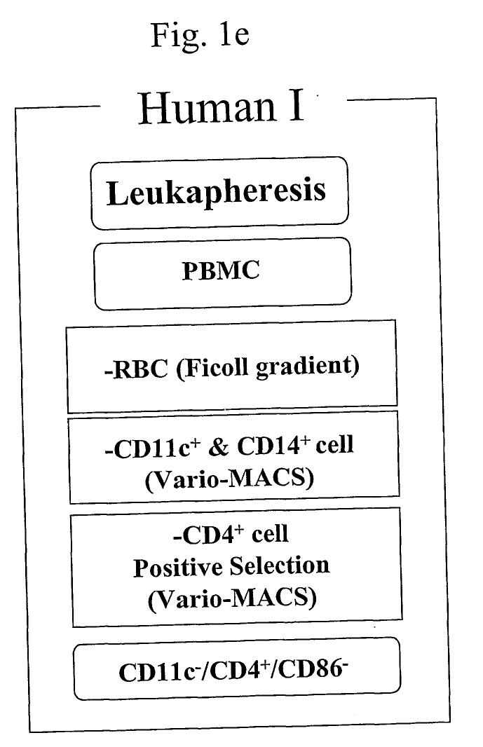

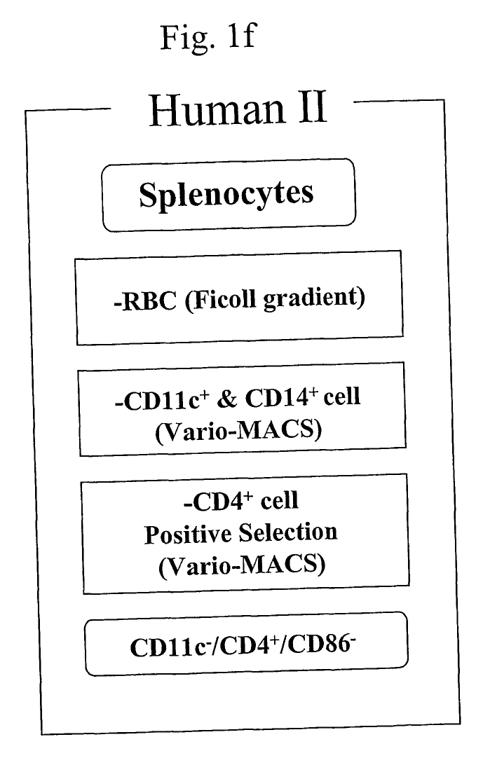

10 mM EDTA was added into the collagenase solution and mixed thoroughly. After centrifugation, the spleen cells were suspended in cold PBS containing 2% FCS, 10 mM HEPES and 10 mM EDTA and then cells with low density were separated by centrifugation in Ficoll-Hypaque (Amersham Pharmacia Biotech, USA). Separated cells were rinsed with PBS twice, resuspended, overlaid on top layer of 17.5% metrizamide (No.M3383; Sigma, USA) solution and then centrifuged to separate cells with low density again. The separated cells were rinsed with MACS solution (PBS containing 0.5% BSA and 2 mM EDTA), counted and reacted with 10 μl/10⁷ cells of magnetized antibodies against CD90, CD19 and NK (Microbeads: No. 130-049-101, 130-049-601, 130-052-501; Miltenyi Biotech, Germany) for 15 min at 6-12° C. and then passed through LS or MS column (No. 130-042-401, 130-042-201; Miltenyi Biotech, Germany). For isolation of the CD11b⁻/CD8a⁺DC, 10 μl/10⁷ cells of antibody against CD8a (No. 130-049-401; Miltenyi Biotech, Germany) was reacted for 15 min at 6-12° C. and then passed through MS column serially. The bound CD11b⁻/CD8a⁺/CD86⁻ DC in column were gathered by washing with 1 ml of MACS solution. The summarized procedure of this example is described on FIG. 1a.

I-2: Isolation of CD11b⁻/CD8a⁺/CD86⁻ Dendritic Cells (DC) (Method 2)

The spleens were removed from ICR or BalB/c mice (Daehan Biolink, Korea), rinsed with PBS in petridish and then collagenase solution (100 units/ml in PBS: Sigma Type IV) was injected into the rinsed spleens with a syringe. After 5 min reaction, the spleens were chopped with a syringe needle for exudation of spleen cells into collagenase solution. Remained spleen cells were separated into collagenase solution by reacting the spleens with collagenase solution in a 50 ml tube for 25 min at RT.

10 mM EDTA was added into the collagenase solution and mixed thoroughly. After centrifugation, erythrocytes were disrupted by reaction in 10 ml of erythrocyte-specific lysis buffer (0.14 M NH₄Cl, 0.02 M Tris-Cl, pH 7.2) for 10 min at RT. Erythrocyte-disrupted spleen cells were resuspended into 5% FBS-RPMI 1640 (Gibco BRL, USA), the media volume was adjusted not to be over 1×10⁸ cells/100 mm dish and then incubated for 90 min at 37° C. After incubation, the loosely attached cells onto plate bottom were discarded by pipetting 9-10 times. Remained loosely attached cells were discarded again in a same manner as previous washing in 10 ml of pre-warmed RPMI 1640 in a 37° C. water bath. 10 ml/dish of pre-warmed 5% FBS-RPMI 1640 was replenished and incubated for 60 min. After incubation, cells were rinsed twice in a same manner as previous washing. After final washing, 10 ml/dish of 5% FBS-RPMI 1640 was refreshed and then incubated for 18-24 hrs. Suspended cells on incubated media were harvested. To harvest loosely attached DC onto plate, cells were rinsed in 5 ml of 5% FBS-RPMI 1640.

The harvested cells were counted, reacted with 10 μl/10⁷ cells of magnetized antibodies against CD90, CD19 and NK (Microbeads: No. 130-049-101, 130-049-601, 130-052-501; Miltenyi Biotech, Germany) for 15 min at 6-12° C. and then passed through LS or MS column (No. 130-042-401, 130-042-201; Miltenyi Biotech, Germany). For isolation of the CD11b⁻/CD8a⁺ DC, 10 μl/10⁷ cells of antibody against CD8a (No. 130-049-401; Miltenyi Biotech, Germany) was reacted for 15 min at 6-12° C. and then passed through MS column serially. The bound CD11b⁻/CD8a⁺ DC in column were isolated by washing with 1 ml of MACS solution (in cold PBS solution containing 2 mM EDTA and 0.5% BSA). The summarized procedure of this example is described in FIG. 1b.

### EXAMPLE II

### Isolation of CD11b⁻/CD8a⁺/CD86⁺ Dendritic Cells (DC) from Mouse Spleen

II-1: Isolation of CD11b⁻/CD8a⁺/CD86⁺ Dendritic Cells (DC) (Method 3)

The spleens were removed from ICR or BalB/c mice(Daehan Biolink, Korea), rinsed with PBS in Petridish and then collagenase solution (100 units/ml in PBS: Sigma Type IV, USA) was injected into the rinsed spleens with a syringe. After 5 min reaction, the spleens were chopped with a syringe needle for exudation of spleen cells into collagenase solution. Remained spleen cells were separated into collagenase solution by reacting the spleens with collagenase solution in a 50 ml tube for 25 min at RT. 10 mM EDTA was added into the collagenase solution and mixed thoroughly. After centrifugation, the spleen cells were resuspended into cold PBS containing 2% FCS, 10 mM HEPES and 10 mM EDTA and then cells with low density were separated by centrifugation in Ficoll-Hypaque (Amersham Pharmacia Biotech, USA). Separated cells were rinsed with PBS twice, resuspended, overlaid on 17.5% metrizamide (No.M3383; Sigma, USA) and then centrifuged to separate cells with low density again. The separated cells were rinsed with MACS solution (PBS containing 0.5% BSA and 2 mM EDTA), counted, reacted with 10 μl/10⁷ cells of magnetized antibodies against CD90, CD19 and NK (Microbeads: No. 130-049-101, 130-049-601, 130-052-501; Miltenyi Biotech, Germany) for 15 min at 6-12° C. and then passed through LS or MS column (No. 130-042-401, 130-042-201; Miltenyi Biotech, Germany). For isolation of the CD11b⁻/CD8a⁺ DC, 10 μl/10⁷ cells of antibody against CD8a (No. 130-049-401; Miltenyi Biotech, Germany) was reacted for 15 min at 6-12° C. and then passed through MS column serially. The bound CD11b⁻/CD8a⁺ DC in column were gathered by washing with 1 ml of MACS solution. The summarized procedures of this invention were described on FIG. 1c.

II-2: Isolation of CD11b⁻/CD8a⁺/CD86⁺ Dendritic Cells (DC) (Method 4)

The spleens were removed from ICR or BalB/c mice (Daehan Biolink, Korea), rinsed with PBS in Petridish and then collagenase solution (100 units/ml in PBS: Sigma Type IV, USA) was injected into the rinsed spleens with a syringe. After 5 min reaction, the spleens were chopped with a syringe needles for exudation of spleen cells into collagenase solution. Remained spleen cells were separated into collagenase solution by reacting the spleens with collagenase solution in a 50 ml tube for 25 min at RT.

10 mM EDTA was added into the collagenase solution and mixed thoroughly. After centrifugation, the spleen cells were washed twice into cold PBS containing 2% FCS, 10 mM HEPES and 10 mM EDTA. After rinse, spleen cells were resuspended in 1 ml/mouse of high density BSA solution (38% BSA) and 5-6 ml of the resuspended solution was aliquot into 15 ml tube. To separate cells with low density, 1-1.5 ml cold RPMI 1640 was overlaid onto the solution delicately and centrifuged. The separated cells were washed twice and counted.

Separated spleen cells were resuspended into 10% FCS-RPMI 1640 (Gibco BRL, USA), the media volume was adjusted not to be over 1×10⁸ cells/100 mm dish and then incubated for 2 hrs at 37° C. After incubation, the loosely attached cells onto dish were detached by pipetting 9-10 times. Remained loosely attached cells were detached again in a same manner as previous washing with 10 ml pre-warmed RPMI 1640 in a 37° C. water bath. 10 ml/dish pre-warmed 10% FCS-RPMI 1640 was replenished and incubated for 18 hrs. Suspended cells on incubated media were harvested. To harvest loosely attached DC onto dish, cells were rinsed in 5 ml of 10% FCS-RPMI 1640.

The harvested cells were counted, reacted with 10 μl/10⁷ cells of magnetized antibodies against CD90, CD19 and NK (Microbeads: No. 130-049-101, 130-049-601, 130-052-501; Miltenyi Biotech, Germany) for 15 min at 6-12° C. and then passed through LS or MS column serially (No. 130-042-401, 130-042-201; Miltenyi Biotech, Germany). For isolation of the CD11b⁻/CD8a⁺ DC, 10 μl/10⁷ cells of antibody against CD8a (No. 130-049-401; Miltenyi Biotech, Germany) was reacted for 15 min at 6-12° C. and then passed through MS column serially. The bound CD11b⁻/CD8a⁺ DC in column were isolated by washing with 1 ml of MACS solution (cold PBS containing 2 mM EDTA, 2% FES). The summarized procedure of this example is described in FIG. 1d.

### EXAMPLE III

### Comparison of Isolating Efficiency and Expression of Cell Surface Proteins of Lymphoid Dendritic Cells (DC)

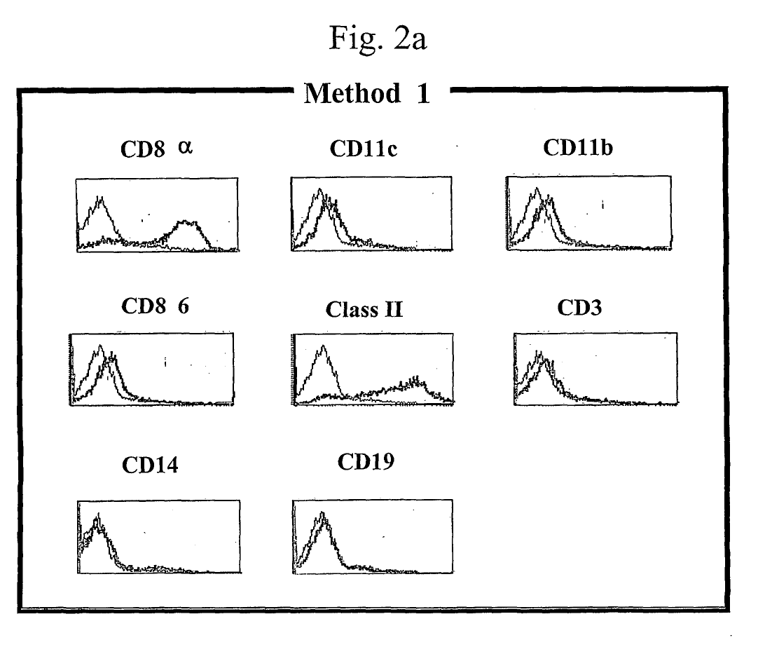

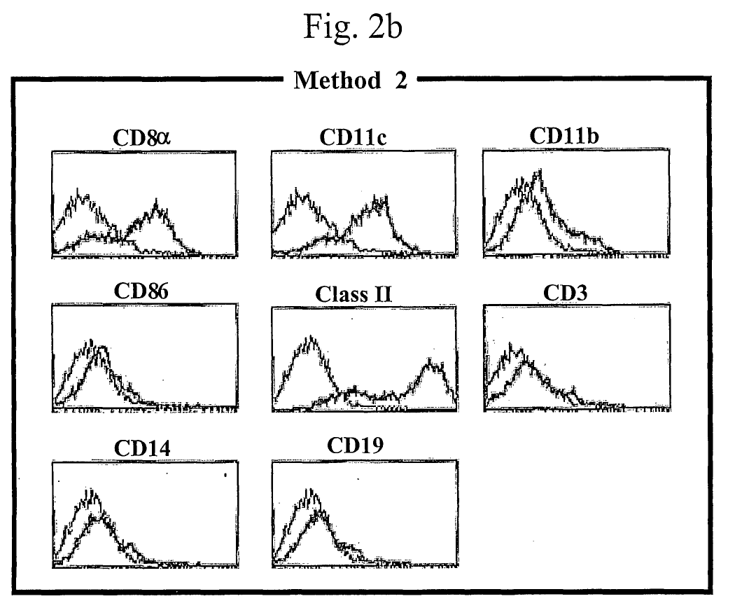

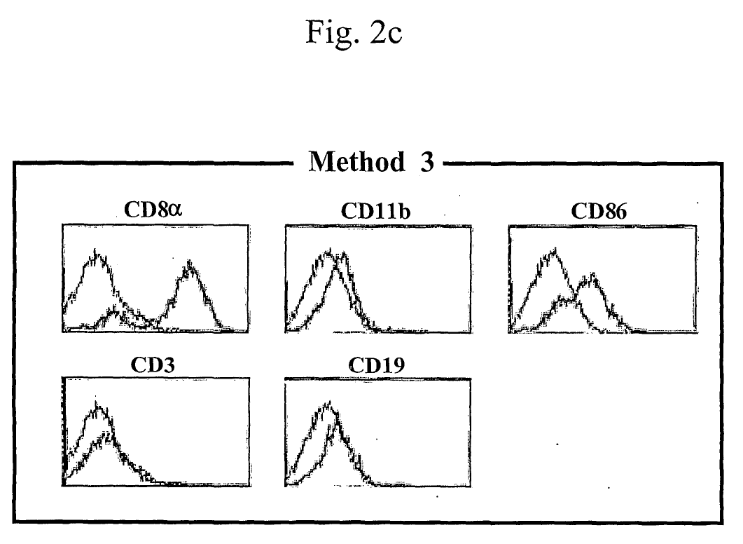

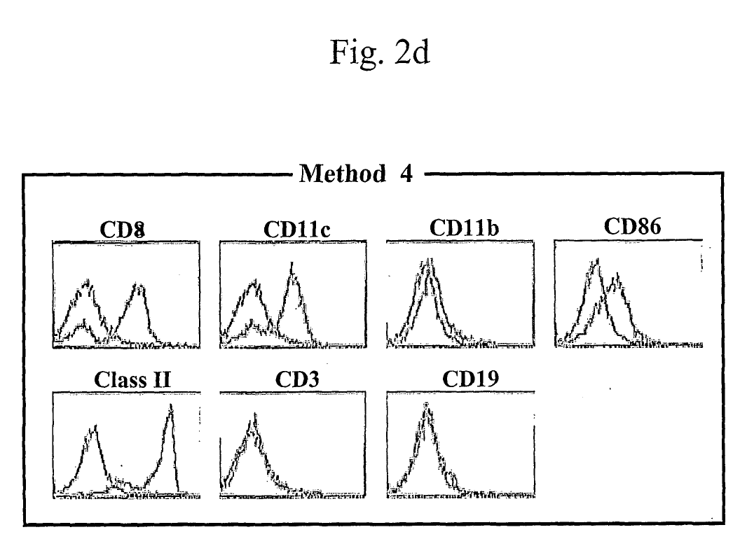

The isolated DC from Example I and II were immunostained with PE (phycoerythrin) and FITC (fluorescein isothiocyanate) labeled monoclonal antibodies (Pharmingen, USA) and assayed as follow: The 2×10⁴ DC from Example I and II were aliquot into each flow cytometry tube (Falcon 2052: Becton Dickinson, USA), added 3 ml of flow cytometry solution (0.2% BSA in cold PBS) and centrifuged. After centrifugation, cells were resuspended into 200 μl of flow cytometry solution, mixed 4 μl of each fluorescence labeled monoclonal antibody and reacted for 30 min at 4° C. Unattached antibodies were discarded by adding 3 ml flow cytometry solution and centrifugation consecutively. Centrifuged cells were resuspended thoroughly into 200 μl flow cytometry solution and analyzed by flow cytometer (FACScalibur flow cytometer; Becton Dickinson, USA) equipped with flow cytometry analyzing program (CellQuest software, USA) (FIG. 2).

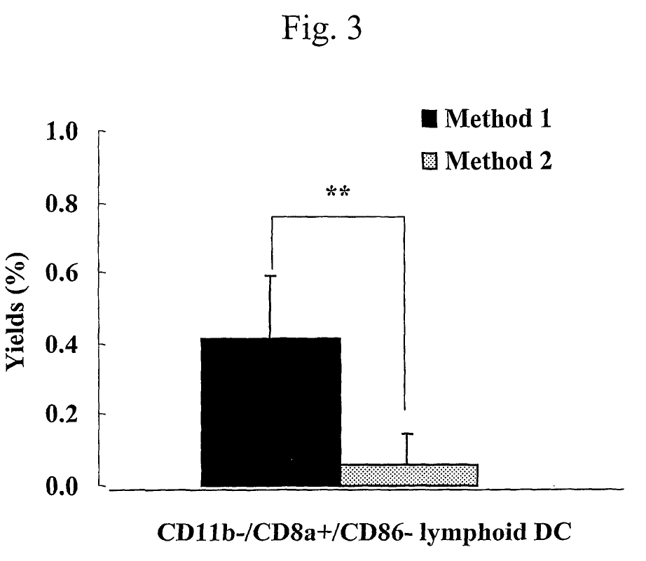

As shown in FIG. 2, the isolated cells were not immunostained against CD3, CD19 and CD14, which are the surface markers of T cells, B cells and monocytes, respectively. This result shows the isolated cells from Examples I and II are DC but not lymphocytes and monocytes. In addition, the low expression of B7 molecules (CD80/CD86), which are expressed on activated antigen presenting cells (APC) shows the DC isolated from Example I are immatured DC subsets rather than fully matured DC. In contrast, the isolated cells from conventional Example II shows high expression level of B7 molecules. Moreover, it is confirmed that 17.5% metrizamide-applied Method 1 described in Example I shows about 7 fold higher isolating efficiency in cell number than the attachment-applied Method 2 (FIG. 3).

### EXAMPLE IV

### Viability of Isolated CD11b⁻/CD8a⁺/CD86⁻ Dendritic Cells (DC) under IFN-γ Treatment

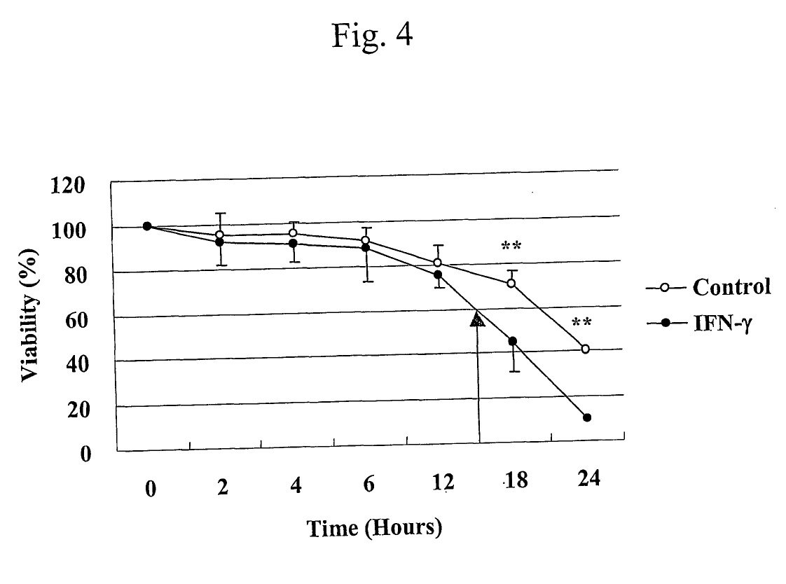

2×10⁶ cells/ml of CD11b⁻/CD8a⁺/CD86⁻ DC, isolated by Method 1 described in Example I, were suspended in 10% FBS-RPMI 1640 (Gibco RBL 31800-022, USA) containing 100 U/ml of IFN-γ (PharMingen 19301T, USA) and incubated for 15 hrs. As shown in FIG. 4, the viability of the isolated cells after 15 hr incubation was 50-60%.

### EXAMPLE V

### Incidence Rate of Spontaneous Diabetes Mellitus (DM) and Determination of Therapeutic Standard of Blood Glucose Level Depending on Age of NOD Mice

7-8 week old female NOD/Ltj mice (Jackson, USA) were fed in a feeding chamber under controlled temperature (23±2° C.) and humidity (55±10%). Mice were housed in cage Myungjin, Korea) not to be over 5 mice/cage under 12 hrs/day artificial light. The water and feed (Samyang-Feed, Korea) were provided ad libitum.

The case of DM was checked weekly on 10:00 AM by measuring weight and blood glucose. Blood, sampled from retinal vein using heparin treated capillary tube (Chase 2051, USA), was measured for blood glucose with blood glucose meter (Glucotrend; Boehringer Mannheim, Germany) and weight was measured with animal balance (Mettler, USA).

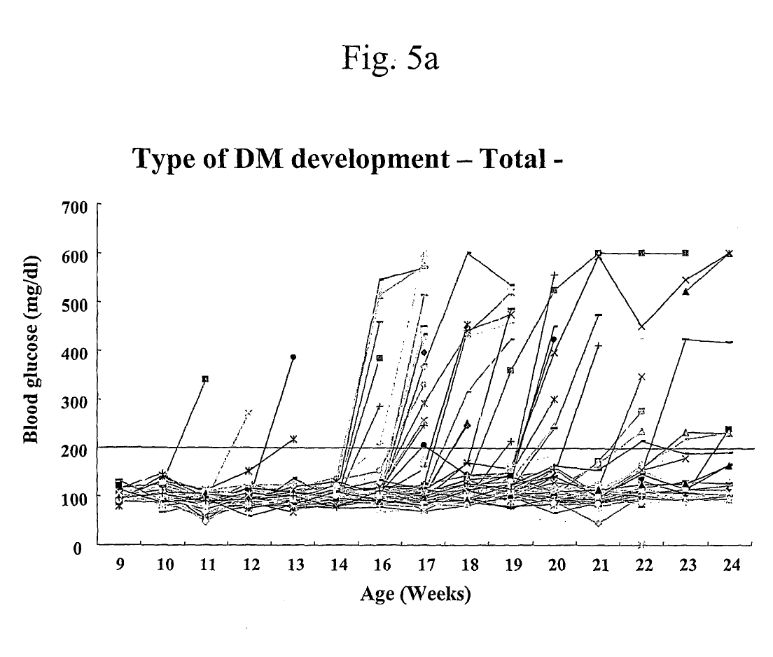

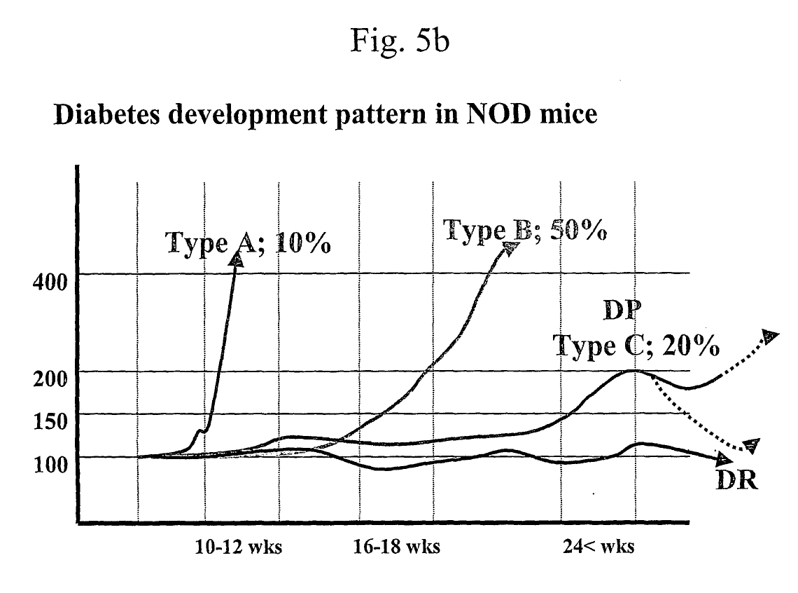

During 24 weeks, 57.75% mice showed diabetic symptoms (67/116 NOD mice), and over than 80% of NOD mice that once blood glucose reached around 200 mg/dl showed severe diabetic progression (FIG. 5a). FIG. 5a is summarized in FIG. 5b. In FIG. 5b, type A mice showed diabetic symptoms at 10-12 week with fast diabetic progression (blood glucose reached to 400 mg/dl in a week), type B showed diabetic symptoms at 16-18 week with somehow slow diabetic progression (blood glucose reached to 400 mg/dl in two weeks) and type C showed diabetic symptoms at 20 weeks or later with very slow diabetic progression (blood glucose reached to 400 mg/dl in 4 weeks or later). Type A, B and C were classified as diabetes-prone (DP) mice and other mice whose blood glucose was below 150 mg/dl even after 24 week were classified as diabetes-resistance (DR) mice.

Hereinafter, all examples were performed with type B mice (16-22 weeks old) referred to FIG. 5b and the DC isolated in Example I-1 were intraperitoneally injected to mice exhibiting high blood glucose (200 mg/dl) as shown in Example VI.

### EXAMPLE VI

### Therapeutic Efficacy of Intraperitoneal Injection of Dendritic Cells (DC) in Diabetic NOD Mice

VI-1: Therapeutic Efficacy of Single Intraperitoneal Injection of Dendritic Cell (DC) Subsets.

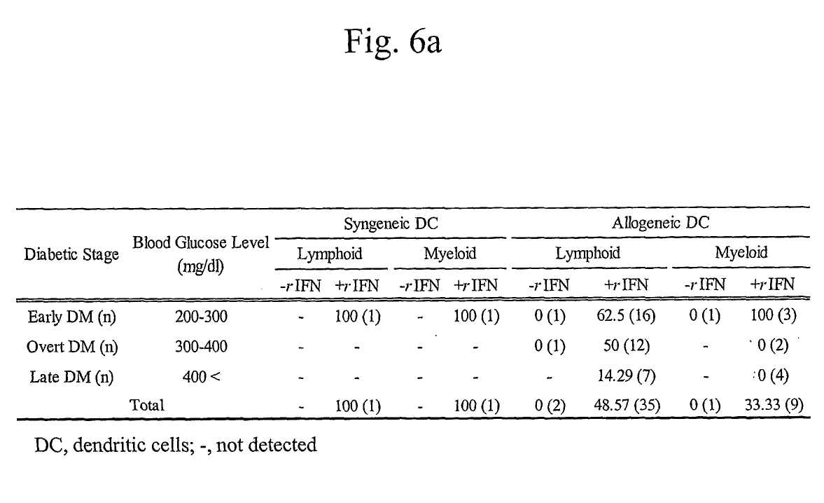

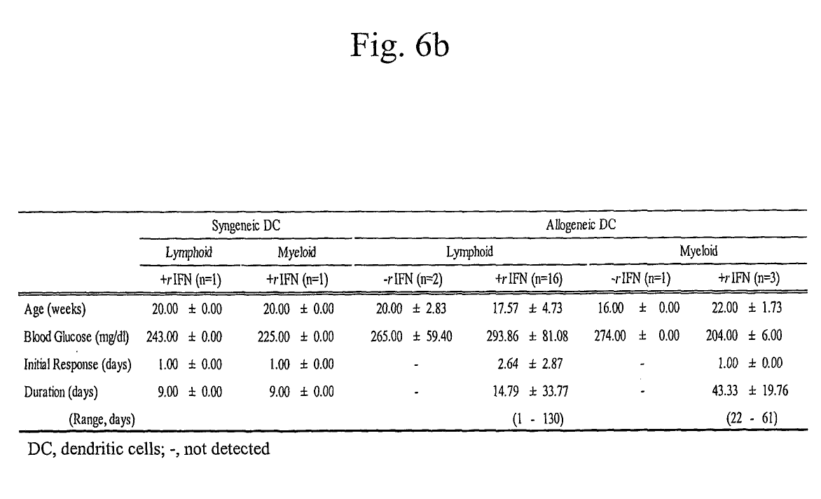

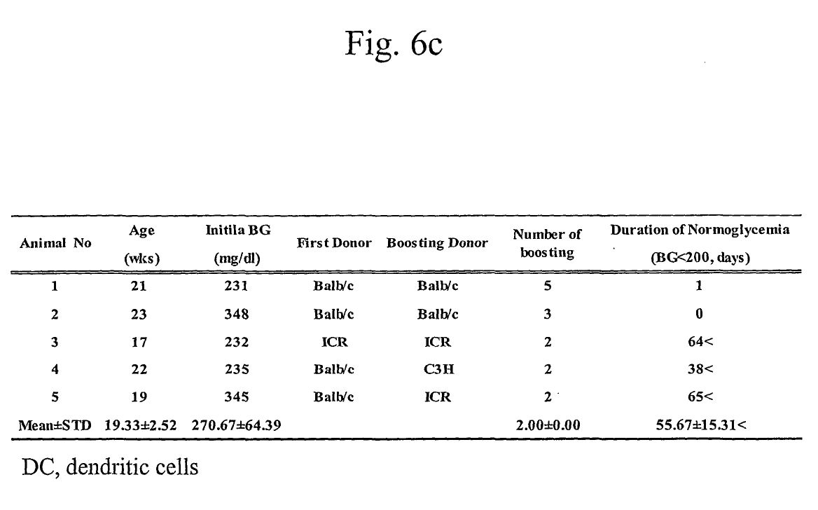

After twice washing of CD11b⁻/CD8a⁺/CD86⁻ DC isolated in Example I-1 or other DC subset with PBS, each 1×10⁶ cells/400 μl of PBS were injected intraperitoneally to diabetic NOD mice. After then, weight and blood glucose were measured as same manner described in Example V for 4 weeks. The therapeutic efficacy was analyzed considering initial response and duration time of response. The initial response indicates the start point when blood glucose was decreased below 200 mg/dl by the injection of DC and duration of therapy indicates the time (days) from initial response to the time when blood glucose was increased over 200 mg/dl (FIGS. 6a and 6b).

FIGS. 6a and 6b show initial response and duration time of response under single injection of DC. Syngeneic DC is one isolated from the same mice with the same major histocompatibility antigen (MHC, e.g., NOD mouse) and allogeneic DC is one isolated from other mice with different MHC (BalB/c mouse).

As shown in FIG. 6, all IFN-γ treated DC subsets showed initial response in early diabetic stage (early DM). Duration time of response was also ranged from 1 to 130 days.

In the case of overt DM and late DM, only IFN-γ treated allogeneic lymphoid DC showed initial response. Especially, IFN-γ treated myeloid DC showed initial response only in mice with early DM but not in overt or late DM, which suggests the different mechanisms between lymphoid DC and myeloid DC. In addition, IFN-γ treated syngeneic DC were shown to be efficient in early DM as well.

These results suggest it would be practicable to control or remove autoimmune T lymphocytes transiently, if DC were differentiated or matured in vitro appropriately.

VI-2: Therapeutic Efficacy of Repetitive Intraperitoneal Injection of allogeneic lymphoid CD11b⁻/CD8a⁺/CD86⁻ DC Subsets

Although single injection of DC showed decreased blood glucose till 130 days depending on mouse, most cases showed transient effect. Therefore, allogeneic lymphoid CD11b⁻/CD8a⁺/CD86⁻ DC, which were evaluated as the most effective DC in single injection, were activated by IFN-γ treatment in vitro, and the IFN-y treated CD11b⁻/CD8a⁺/CD86⁻ DC were injected repeatedly (boosting) for prolonged or life-time therapeutic effect as followed procedures:

IFN-α treated allogeneic lymphoid CD11b⁻/CD8a⁺/CD86⁻ DC, isolated from ICR or BalB/c mice, were injected into 17-23 week old 5 NOD mice with DM symptoms in a same manner used in Example VI-1 and boosted at the point when blood glucose increased or previously. As shown in FIG. 6c, boosting with the same allogeneic DC showed very short effect. However, trans-allo-DC treatment (BalB/c to ICR, BalB/c to C3H or ICR to BalB/c) showed prolonged decrease in blood glucose just by double boosting. In FIG. 6c, the enough effect just with DC isolated from ICR mice can be explained as trans-allo-DC effect because ICR mice are outbred strain.

### EXAMPLE VI-3

### Therapeutic Efficacy by Treatment of IFN-γ Alone

As shown in Example VI-1, IFNγ was shown to be prerequisite for therapy of DM using DC. Therefore, therapeutic function of IFN-γ alone on DM was evaluated. NOD mice used in Example V were injected with 600 U/mouse of IFN-γ, but all mice were not shown any therapeutic efficacy except for 1 mouse which died after showing transient therapeutic efficacy. This result indicates that the therapeutic effect of IFN-γ treated DC on DM, which is described in Example V-1, is not due to IFN-γ itself and is resulted from local immune response rather than systemic immunity.

### EXAMPLE VII

### Histopathological Validation of DC on DM Therapy

VII-1: Validation by Hematoxylin and Eosin (H&E) Staining of Pancreas

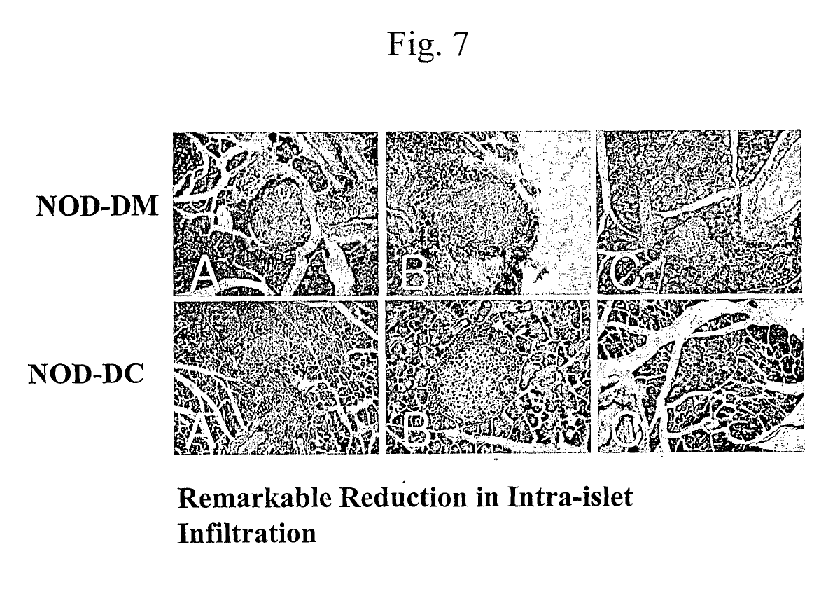

The NOD mice with early DM used in Example V were injected intraperitoneally with allogeneic IFN-γ treated lymphoid CD11b⁻/CD8a⁺/CD86⁻ DC (1×106 cells/mouse) as described in Example VI-1 and mice were decapitated, and the pancreas was removed therefrom 4 weeks after DC injection. Removed pancreas were fixed in 10% neutral formalin for 24 hrs, dehydrated with alcohol, embedded in paraffin and sectioned in 4 μm thickness with microtome (Zeiss Super Cut 2050, Germany). Then, section was stained with hematoxylin and eosin, and the grade of insulitis was evaluated under light microscope (Nicon, Japan) observation (FIG. 7). Insulitis was graded as follows: insulitis score 0, no lymphocytes infiltration; 1, less than 25% of islet were infiltrated with lymphocytes; 2, 25-50 of islet were infiltrated with lymphocytes; 3, 50-75 of islet were infiltrated with lymphocytes; and 4, more than 75% of islet were infiltrated with lymphocytes.

NOD-DM in FIG. 7 indicates the severity of insulitis in DC-untreated mouse showing early insulitis (A, insulitis score 1) in 1 week after DM development, fast progressed insulitis (B, insulitis score 3) and islet fully filled with T lymphocytes showing destructive all β-cells (C, insulitis score 4). In DC-treated islets, the severity of insulitis was negligible since traces of T cell infiltration was slightly observed without severe insulitis (A, insulitis score 1). Although the peripheral region of islets treated by DC was shown to be infiltrated by T cell in some mice (B, insulitis score 2), the central region of the islet was shown normal appearance (C, insulitis score 0) compared to control with hyperglycemia. In some cases, it was observed that the traces of T cell infiltration remained, but they were disappeared to exhibit appearance of normal islets.

VII-2: Validation by Insulin Immunostaining of Islets

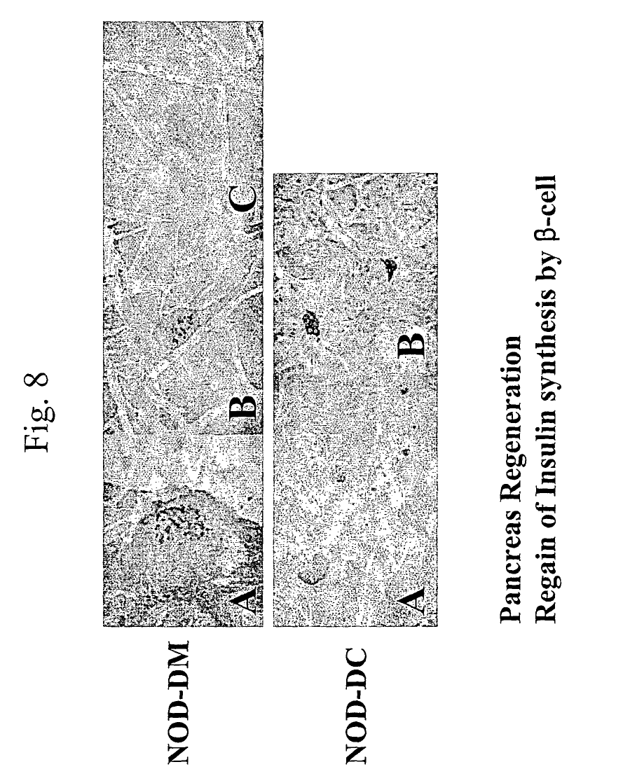

For immunohistochemcal analysis of insulin secretion, removed islets were fixed in 10% neutral formalin for 24 hrs, dehydrated by alcohol and embedded in paraffin. Then, 4 μm tissue section was prepared for immunohistochemical staining of insulin-secreting cells (islet β-cells) with avidin-biotin complex as below: Sectioned pancreatic tissue was reacted with solution containing methanol containing 1% hydrogen peroxide for 30 min to inactivate intrinsic hydrogen peroxidase, antibody against insulin (1:400, guinea pig anti-porcine insulin, Dako Co., Denmark) was treated and probed for 24 hrs in 4° C. humid chamber. Biotinylated anti-guinea pig IgG (Vector, USA) was probed as secondary antibody and horseradish peroxidase labeled avidin solution (Vector, USA) was reacted. Each stain was performed in 0.1 M PBS containing 10% goat serum (S-2007, Sigma, USA). After antigen-antibody probing, 0.3 mg/ml of 3,3′-diaminobenzidine (DAB, D8001, Sigma, USA) and 0.003% H₂O₂ were added for color development. The reaction was stopped when appropriate color developed under light microscope observation. The slides were counter-stained with Meyer's hematoxylin (FIG. 8).

In FIG. 8, 3 pictures of NOD-DM showed insulin-immunostaining results of control NOD mouse islets without DC-injection, and 2 pictures of NOD-DC showed insulin-immunostaining results of diabetic NOD mice whose blood glucose was recovered to normal level by injection of IFN-γ treated allogeneic lymphoid CD11b⁻/CD8a⁺/CD86⁻ DC with showing the normal blood glucose level during 20 days.

As shown in FIG. 8, DC-untreated control mice showed most islets were destroyed by insulitis, and so the insulin reactivity was weak (FIG. 8, NOD-DM-A, -B) or undetectable (FIG. 8, NOD-DM-C). In contrast, pancreatic section of DC-treated NOD mice showed remained not-destructed part of islets with normal insulin reactivity (FIG. 8, NOD-DC-A, -B). In addition, DC-treated mice showed a number of insulin-positive small islet clusters around the pancreatic ducts and exocrine portion of pancreas, which indicate maintenance of new islet formation after DC treatment (FIG. 8, NOD-DC-A).

### EXAMPLE VIII

### In Vivo Migration Study of DC or Autoimmune T Lymphocytes by Tracing with CMTMR or CMFDA

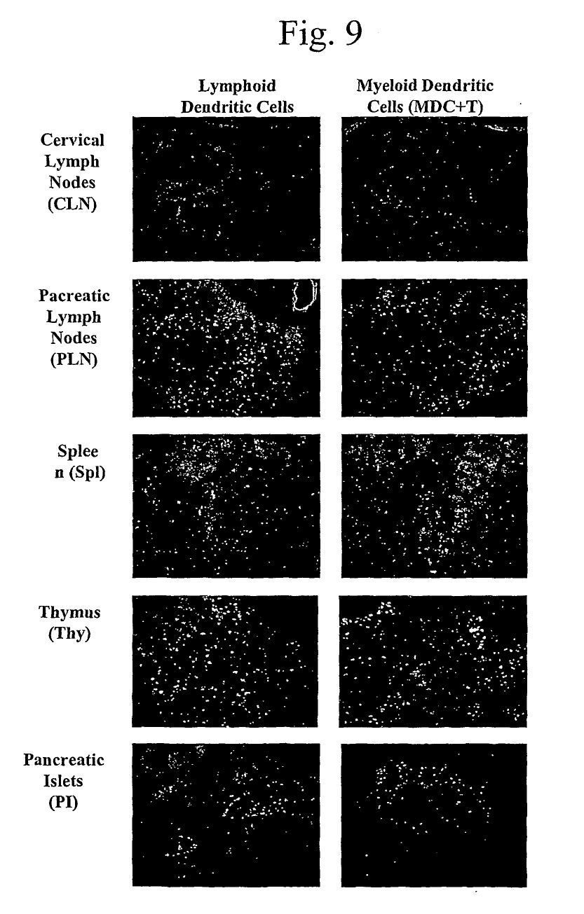

2 μM CMTMR (No. C-2926, Molecular Probe, USA) and CMFDA (No. C-2925, Molecular Probe, USA) were used to trace in vivo migration of cells after dilution in serum- or other ingredient-free RPMI 1640 immediately before use. T lymphocytes were isolated from overt diabetic NOD mice with nylon wool and lymphoid or myeloid DC isolated by Method 1 of Example I was used. For staining, 100 μl/10⁶ cells of CMTMR or CMFDA solution was added, reacted for 15 min at 37° C. and washed twice. Thereafter, cells were resuspended with fresh RPMI 1640 and incubated for 30 min at 37° C. for converting incorporated CMTMR or CMFDA to impermeable molecules. Overt DM-developed NOD mouse was slightly anesthetized with ether, the 3×10⁶ CMFDA-stained autoimmune T lymphocytes in 200 μl of PBS were injected intravenously via tail vein and 1×10⁶ CMTMR-stained lymphoid or myeloid DC in 400 μl of PBS were injected to NOD mice intraperitoneally. The mice were sacrificed 48 hrs later, pancreas were removed, frozen immediately in tissue freezing media (Jung 0201 08926, Germany,). Frozen tissues were sectioned in 5 μm thickness by Cryostat (CM1510-3, Leica, Germany), and observed under confocal microscopy (Bio-Rad, MRC 1024ES, Hercules, USA) directly (FIG. 9).

In FIG. 9, LDC+T showed NOD mice injected with lymphoid DC and autoimmune T lymphocytes, and MDC+T showed NOD mice injected with myeloid DC and autoimmune T lymphocytes. Each pictures showed extent of fluorescence on the slice of CLN (cervical lymph nodes), DLN (deep lymph nodes), PI (pancreatic islets), Spl (spleen), PLN (pancreatic lymph nodes) or Thy (thymus). As shown in FIG. 9, injected CD11b⁻/CD8a⁺/CD86⁻ DC and IFN-γ treated CD11b⁻/CD8a⁺/CD86⁻ DC for 15 hrs were found most abundantly in pancreatic lymph nodes and also found abundantly at spleen, thymus, islet and distant tissue from peritoneum on order. The distribution of autoimmune T lymphocytes was also found to have identical pattern to injected DC, which demonstrates that injected DC works mainly at autoimmune disease-occurred tissue and thymus.

### EXAMPLE IX

### Culture of Pancreatic Lymph Node Cells Specific to Islet Antigen and Quantification of Cytokine Expression

In DM-cured NOD mice by single injection of IFN-γ stimulated CD11b⁻/CD8a⁺ ICR mouse DC as described in Example VI-1, changes of autoimmune response against islet β-cell were checked as follows:

IX-1: Proliferation of Lymph Node Cells Specific to Islet Antigen and Isolation of Islets

Lymph node cells were isolated from pancreatic lymph nodes extracted from NOD mice with early DM to which 1×10⁶ of IFN-γ treated CD11b⁻/CD8a⁺ DC were injected. In addition, from DC-untreated NOD mice with early DM, lymph node cells were isolated. Thereafter, isolated cells were suspended with 5% FBS-DMEM and 5×10⁴ cells/well were aliquot into each well.

Separately, β-cells from pancreatic islets were isolated from NOD mice and ultrasonicated, thereafter, the extract amount of 2.5×10⁴ or 5×10⁴ cells (CEQ: cells of equivalent) was added into each well as islet antigen and incubated for 96 hrs. IFN-γ (indicator cytokine of activated Th1) and IL-4 (indicator cytokine of activated Th2) in supernatant were measured by sandwich ELISA method as Example IX-2.

The islets isolated above was obtained as below: NOD mouse was anesthetized by intraperitoneal injection of 1 ml/100 g of 20% urethane, the peritoneum was surged and pancreas was removed after injection of collagenase P into common pancreatic duct. The removed pancreas was incubated for 10 min at 37° C. and effused tissues from the digested pancreas were harvested. Harvested tissues were washed twice with PBS by centrifugation, resuspended evenly in Ficoll with a density of 1.086 g/ml and overlaid with Ficoll with a density of 1.076 and 1.053 g/ml serially. Thereafter, tube was centrifuged for 10 min at 800×g in a refrigerated centrifuge, and islets between density of 1.076 and 1.053 were taken carefully, washed twice with PBS, incubated for 24 hrs in 5% CO₂ incubator at 37° C. and handpicked the cultured islets under microscope.

IX-2: Quantification of Cytokines by ELISA

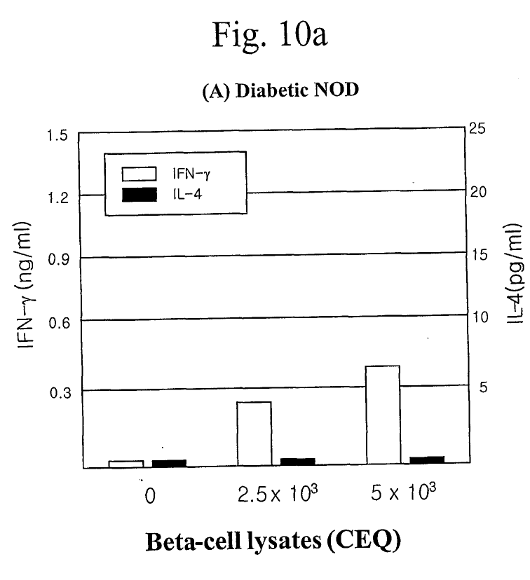

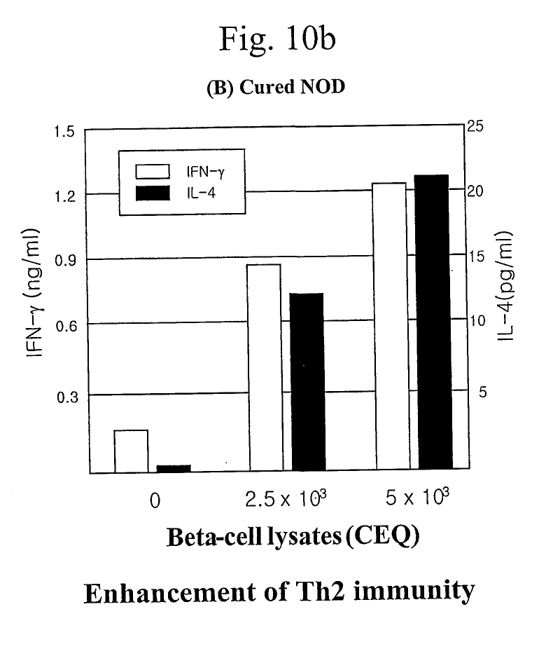

IL-4 and IFN-γ in culture supernatant were measured by sandwich-ELISA method using the supplied materials and matched antibody pairs from Endogen as below: 96-well culture plate was coated with 100 μl (2 μg/ml) of coating antibody (Endogen, USA) for 10-14 hrs at RT and washed. After then, 200 μl of analytical buffer (PBS with 4% BSA, pH 7.2-7.4) was added and reacted for 1 hr at RT. After 3 time washing, 50 μl of diluted supernatant and standard solution were added into each wells in a duplicate manner, 50 μl (250 μg/ml) of biotin-labeled secondary antibody was added and incubated for 1-2 hrs. Reacted well was washed, 100 μl of HRP-fused streptavidin (1:12,000, Endogen, USA) was added and reacted for 30 min in darkness. Each well was washed with washing buffer, reacted with TMB substrate (Sigma T-3405, USA) for 30 min and reaction was stopped by addition of 2 M H₂SO₄. The amount of cytokine was calculated with OD₅₇₀ comparing to standard solution (FIG. 10a and b).

FIG. 10a showed cytokines produced from the lymph node cells from DC-untreated NOD mice with early DM as used Example V and FIG. 10b showed cytokines produced from the lymph node cells from DM-cured mice by single injection of DC. In DC-untreated control, just IFN-γ but not IL-4 was detected in lymph node cell culture under islet antigen (FIG. 10a). While in DM-cured NOD mouse by DC-injection, as amount of islet antigen was increased, significant increase of IFN-γ and IL-4 was detected (FIG. 10b).

These results suggest the therapeutic application on DM in which progression of insulitis is inhibited by DC-treatment, possibly leading to development of Th1 autoimmune T lymphocytes into Th2 lymphocytes or inactivation of T lymphocytes by newly developed islet antigen-specific Th2 lymphocytes.

### EXAMPLE X

### Morphological Study of CD11b⁻/CD8a⁺ DC Using Transmission Electron Microscope (TEM)

CD11b⁻/CD8a⁺/CD86⁻ DC were isolated from ICR mice by the same procedures as Example I, washed with PBS, pre-fixed for 2 hrs in 2% paraformaldehyde/2.5% glutaraldehyde solution (4° C., pH 7.2) dissolved and washed with 0.1 M PBS 3 times. Washed DC were post-fixed for 1 hr in 1% OSO₄ solution (4° C., pH 7.2) in PBS. Fixed DC were washed several times in PBS and dehydrated in series of graded ethanol dilutions (30, 50, 70, 80, 90, 95% once each and absolute alcohol twice). Dehydrated specimen was cleaned by propylene oxide, embedded in Epon-Araldite solution (Poly/Bed 812 Embedding Media, Polysciences Inc.) and heatpolymerized for 28 hrs at 60° C.

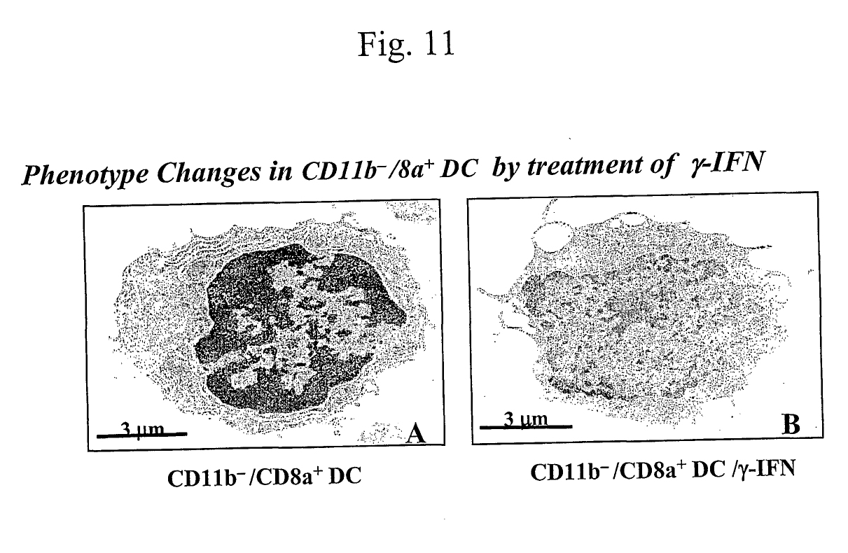

Embedded tissue was sectioned in semithin section thickness by LKB-V ultramicrotome, stained with 1% toluidine blue dissolved in 1% vorax on 60° C. heated hot plate and observed under light microscope. Thereafter, thin section was prepared, bound on nickel grid, stained with uranyl acetate mixed with lead citrate and examined under transmission electron microscope (JEOL co., Japan) at 80 kV (FIG. 11).

In FIG. 11, panel A shows the picture of IFN-γ untreated CD11b⁻/CD8a⁺/CD86⁻ DC and panel B shows the picture of IFN-γ treated CD11b⁻/CD8a⁺ DC. Endoplasmic reticulum (ER), nuclear membrane and chromosome were well visualized same as immediately isolated DC in IFN-γ untreated DC, but disappearance of ER structure, change of plasmasytoid structure, indistinctness of nuclear membrane, loose chromosomes and significant increase of dendrites were observed in IFN-γ treated DC (FIG. 11). These morphological changes shown in normally developed DC indicate that IFN-γ activates undeveloped lymphoid DC to develop into finally developed DC as activated myeloid DC in spite of different origin.

### EXAMPLE XI

### Isolation of CD11c⁻/CD4⁺/CD86⁻ DC from Human

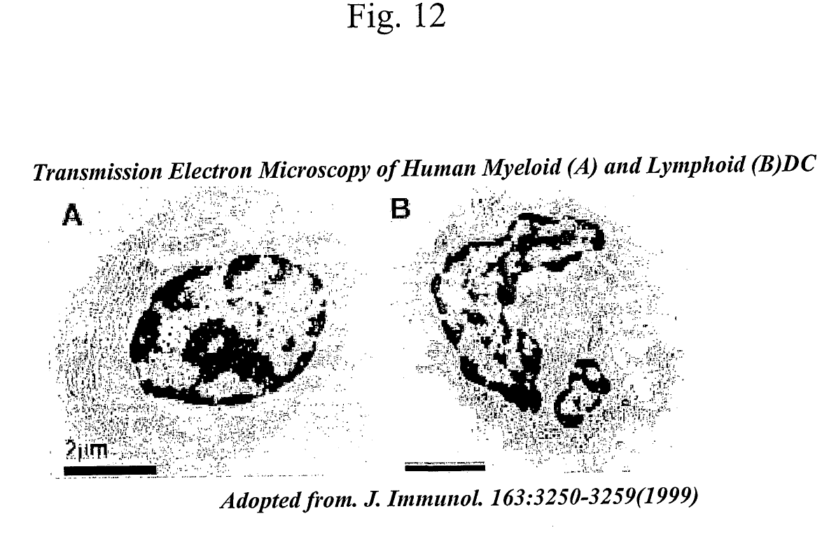

Mouse CD11b⁻/CD8a⁺ DC subset was not found in human. In this invention, human CD11c⁻ DC was considered as mouse CD11b⁻/CD8a⁺ DC on basis of below: Panel A of FIG. 12 showed CD11c⁻ DC isolated from human peripheral blood and panel B showed morphological feature of CD11c⁻ DC. Electron microscopic feature of CD11c⁻ DC on FIG. 12, cited from a published paper (J. Immunol. 163:3250-3259(1999)), showed similar features with CD11b⁻/CD8a⁺ DC in development of ER and shape of chromosomes (FIG. 12, A). This hypothesis is additionally supported by a paper showing the possibility of CD11c⁻ DC to regulate immune responses as a lymphoid DC (O'Doherty et al., Immunology, 82:487-493(1994)) On the base of the hypothesis, CD11c⁻ DC were isolated from human blood as below (FIGS. 1e-1f):

XI-1: Isolation from PBMC

CD11c⁻ DC were isolated from human blood using DC isolating kit (No. 468-01) purchased from Miltenyi Biotech: Concentrated leukocytes acquired from leukapheresis of normal human were diluted in 3 time volume of PBS, Ficoll-Hypaque (10 ml/30 ml of diluted leukocytes) was added, centrifuged for 30 min at RT with 2000 g, and floating cells in the middle layer were harvested. Thereafter, harvested cells were washed 3 times with PBS at serially decreased speed (1600 g, 1200 g and 900 g) for 5 min. Washed peripheral blood mononuclear cells (PBMC) were counted, and CD11c⁻ DC subset was isolated from 2.5-3×10⁸ PBMC as below (FIG. 1e).

The 2.5-3×10⁸ PBMC were washed with 20 ml MACS solution (2 mM EDTA, 0.5% BSA in cold PBS). After then, MACS solution was added to be 1.2 ml in total volume, cells were resuspended evenly, 0.4 ml FcR-blocking solution and 0.4 ml heptane-Ab cocktail from the blood DC isolating kit (No. 468-01, Miltenyi Biotech) were added, and the mixture was reacted for 20 min at 4° C. Cells were washed twice in 40 ml MACS solution, centrifuged, and all supernatant was sucked by vacuum pump clearly in each centrifuging steps. MACS solution was added to be 3.6 ml in total volume, resuspended evenly, 0.4 ml MACS anti-heptane bead solution was mixed well and reacted for 20 min at 4° C. CS column kept in refrigerator was assembled into the MACS separator (VarioMACS, No. 130-090-282, Miltenyi Biotech) and washed with 60 ml MACS solution in 4° C.

2 ml of reacted cells were passed through CS column, washed with 30 ml MACS solution and passed cells were gathered for next procedure. Cells were counted, centrifuged, and supernatant was discarded. MACS solution containing 15 μg of CD11c antibody (1 μg/μl, BD science 30480D, USA) was added to be 10⁷ cells/70 μl, and additional 70 μl of CD14 antibody was added, mixed evenly and reacted for 20 min at 4° C. After twice washing with 3 ml MACS solution, washed cells were mixed in MACS solution to be 70 μl in volume, and reacted with 30 μl of magnetic beads labeled with mouse IgG-specific antibody (Goat Anti-Mouse IgG MicroBeads: No. 130-048-401, Miltenyi Biotech) for 15 min at 4-6° C. After then, 3 ml MACS solution added and centrifuged to discard unbound cells.

Cells were diluted in 0.5 ml MACS solution, passed through MS column to discard magnetic-labeled cells. MS column was washed 3 times with MACS solution and non-magnetized cells were separated. Centrifuged cells were mixed in MACS solution to be 0.2 ml in volume, 0.2 ml anti-CD4 magnetic beads from the blood DC isolating kit (No. 468-01, Miltenyi Biotech) was added and reacted for 30 min at 4° C. Unbound magnetic beads were removed by centrifugation in additional 8 ml MACS solution and bead-unbound cells, resuspended in 0.5 ml MACS solution, were removed through MS column. Column was washed with 0.5 ml MACS solution 3 times and column was disassembled from MACS separator (VarioMACS, No.130-090-282, Miltenyi Biotech) Remained cells in column were gathered into 15 ml centrifuge tube by adding 1 ml MACS solution and pushing the column with syringe. Remained non-magnetized cells were removed through MS column again. After 3 time-washing with 0.5 ml MACS solution, magnetized cells in MS column were separated by addition of 1 ml MACS as described previous. Finally, cells were centrifuged and purity of CD11c⁻/CD4⁺/CD86⁻ DC was checked by fluoro-cytometry.

XI-2: Isolation from Spleen Cells

CD11c⁻ DC were isolated from human spleen cells using DC isolating kit (No. 468-01) purchased from Miltenyi Biotech with modified procedures:

Human spleen cells were released from spleen by treatment of collagenase, centrifuged in standard Ficoll condition used for PBMC isolation, and took the cells from boundary line and washed with PBS. Among them, 3×10⁸ cells were washed into 20 ml MACS solution (2 mM EDTA, 0.5% BSA in cold PBS). MACS solution was replenished to be 1.2 ml in total volume, mixed evenly and CD11c⁻/CD4⁺/CD86⁻ DC were isolated as described previous XI-1 (FIG. 1f). Even in the case without procedure for discarding CD11c⁺ cells, high purity (86%) of CD11c⁻/CD4⁺/CD86⁻ DC subset was isolated.

### EXAMPLE XII

### Therapeutic Efficacy of DC on Type I DM Patient

Isolated CD11c⁻/CD4⁺/CD86⁻ DC are treated with IFN-γ as described in Example IV. Thereafter, culture media of CD11c⁻/CD4⁺ DC is precipitated by centrifugation, supernatant is discarded and added with saline. Prepared DC are injected to type I DM patient intraperitoneally. And then, therapeutic effect of DC is evaluated based on blood glucose level,

### EXAMPLE XIII

### Therapeutic Effect of DC in Rheumatoid Arthritis-Induced Mice by Collagen XIII-1: Induction of Rheumatoid Arthritis by Collagen in DBA/1 Mice 5-6 week old male DBA/1 mice (Jackson Laboratory, USA) are used. 2 mg/ml of bovine type II collagen is added into 0.05 M acetate solution and dissolved for 24 hrs at 4° C. on a stirrer. Dissolved collagen is mixed with same amount of complete Freund's adjuvant (CFA) and 100 μg/mouse of collagen will be injected intravenously at the base of tail. After 3 weeks, 100 μg of collagen/mouse in incomplete Freund's adjuvant (IFA) is injected at the base of tail. For synchronous onset of arthritis, 40 μg LPS is injected intraperitoneally 4 weeks after first collagen injection (SH Kim, et al., J. Immunol., 166:3499-3505(2001)).

XIII-2: Therapeutic Effect of DC Subset

CD11b⁻/CD8a⁺/CD86⁻ DC is isolated from spleen of normal DBA/1 mouse as Example I, treated with IFN-γ as Example II and therapeutic effect on arthritis is checked after injection of CD11b⁻/CD8a⁺ DC in leg joint into mice used in Example XII-1.

Therapeutic effect of DC on arthritis is evaluated by macroscopic score ranging from 0 to 4 as below (S H Kim, et al., J. Immunol., 166:3499-3505(2001)); 0: without edema or swelling; 1: trivial edema or swelling on digit or ankle joint partially; 2: trivial edema or swelling from ankle joint to digit overall; 3: significant edema and swelling from ankle joint to digit; and 4: severe edema and swelling from ankle joint to digit especially with deformity or ankylosis on ankle or digit.

Mean arthritis index=total scores of 4 paws in all mice/total mice

Paws with arthritis (%)=all paws with score over 2/all paws×100

Severity of edema=thickness of 4 paws of all mice/number of mice

XIII-3: Histologic Examination for Therapeutic Effect of DC

Ankle joint, freshly dissected from CD11b⁻/CD8a⁺ DC treated mouse used in Example XIII-2, is fixed in 10% neutral formalin solution for 24 hrs, decalcified in 15% EDTA and 30% glycerin, dehydrated in series of gradient alcohol, embedded in paraffin, sectioned in thickness of 5 μm, stained with hematoxylin and eosin (H&E), and therapeutic effect is evaluated considering infiltration of lymphocytes and bone erosion (SH Kim, et al., J. Immunol., 166:3499-3505(2001)).

### EXAMPLE XIV

### Therapeutic Effect of DC on Rheumatoid Patients

CD11c⁻/CD4⁺/CD86⁻ DC isolated from Example X is treated with IFN-γ as described in Example IV. Then, culture media of CD11c⁻/CD4⁺ DC is centrifuged, supernatant is discarded, and saline is added. Prepared CD11c⁻/CD4⁺ DC is injected into joint of rheumatoid patients. Thereafter, therapeutic effect of CD11c⁻/CD4⁺ DC is evaluated.

Having described a preferred embodiment of the present invention, it is to be understood that variants and modifications thereof falling within the spirit of the invention may become apparent to those skilled in this art, and the scope of this invention is to be determined by appended claims and their equivalents.

What is claimed is:

1. A pharmaceutical composition for immunotherapy of autoimmune disease, which comprises (a) a therapeutically effective dose of maturated dendritic cells and (b) a pharmaceutically acceptable carrier.

2. The pharmaceutical composition according to claim 1, wherein the autoimmune disease is one selected from the group consisting of type I diabetes, rheumatoid arthritis, multiple sclerosis, systemic lupus erythematosus, Sjogren's syndrome, scleroderma, polymyositis, chronic active hepatitis, mixed connective tissue disease, primary biliary cirrhosis, pernicious anemia, autoimmune thyroiditis, idiopathic Addison's disease, vitiligo, gluten-sensitive enteropathy, Graves' desease, myasthenia gravis, autoimmune neutropenia, idiopathic thrombocytopenia purpura, cirrhosis, pemphigus vulgaris, autoimmune infertility, Goodpasture's disease, bullous pemphigoid, discoid lupus, ulcerative colitis and dense deposit disease.

3. The pharmaceutical composition according to claim 2, wherein the autoimmune disease is type I diabetes or rheumatoid arthritis.

4. The pharmaceutical composition according to claim 1, wherein the maturated dendritic cells are prepared by treating immature dendritic cells with cytokine selected from the group consisting of IFN-γ, TNF-α, TGF-β, IL-4, IL-10 and combinations thereof.

5. The pharmaceutical composition according to claim 1, wherein the maturated dendritic cells inhibit autoimmune response through converting autoimmune Th1 lymphocytes into Th2 lymphocytes or generating new Th2 lymphocytes.

6. The pharmaceutical composition according to claim 1, wherein the dendritic cells are isolated from human organ, tissue, bone marrow or blood.

7. The pharmaceutical composition according to claim 6, wherein the dendritic cells are allogeneic dendritic cells.

8. The pharmaceutical composition according to claim 7, wherein the dendritic cells are lympoid dendritic cells.

9. The pharmaceutical composition according to claim 8, wherein the lymphoid dendritic cells are CD11c⁻/CD4⁺ dendritic cells.

10. A method for immunotherapy of autoimmune disease comprising the steps of:
(a) preparing maturated dendritic cells; and
(b) administering into mammals a pharmaceutical composition containing (i) a therapeutically effective dose of the maturated dendritic cells and (ii) a pharmaceutically acceptable carrier.

11. The method according to claim 10, wherein the autoimmune disease is one selected from the group consisting of type I diabetes, rheumatoid arthritis, multiple sclerosis, systemic lupus erythematosus, Sjogren's syndrome, scleroderma, polymyositis, chronic active hepatitis, mixed connective tissue disease, primary biliay cirrhosis, pernicious anemia, autoimmune thyroiditis, idiopathic Addison's disease, vitiligo, gluten-sensitive enteropathy, Graves' disease, myasthenia gravis, autoimmune neutropenia, idiopathic thrombocytopenia purpura, cirrhosis, pemphigus vulgaris, autoimmune infertility, Goodpasture's disease, bullous pemphigoid, discoid lupus, ulcerative colitis and dense deposit disease.

12. The method according to claim 11, wherein the autoimmune disease is type I diabetes or rheumatoid arthritis.

13. The method according to claim 10, wherein the maturated dendritic cells are prepared by treating immature dendritic cells with cytokine selected from the group consisting of IFN-γ, TNF-α, TGF-β, IL-4, IL-10 and combinations thereof.

14. The method according to claim 10, wherein the maturated dendritic cells inhibit autoimmune response through converting autoimmune Th1 lymphocytes into Th2 lymphocytes or generating new Th2 lymphocytes.

15. The method according to claim 10, wherein the dendritic cells are isolated from human organ, tissue, bone marrow or blood.

16. The method according to claim 15, wherein the dendritic cells are allogeneic dendritic cells.

17. The method according to claim 16, wherein the dendritic cells are lympoid dendritic cells.

18. The method according to claim 17, wherein the lymphoid dendritic cells are CD11c⁻/CD4⁺ dendritic cells.

19. The method according to claim 10, wherein the method further comprises the step (c) of boosting with trans-allo-dendritic cells.
